SEQUENCE LISTING (18 sequences)

SEQ ID NO: 1 - DNA, 30 bp, COVID19
agggtaaaccaaatacctggtgtatacgtt

SEQ ID NO: 2 - DNA, 27 bp, COVID19
agggttaaaccaccgcctggagatcaa

SEQ ID NO: 3 - DNA, 30 bp, COVID19
agggtccttaacttgcctggttgtgatggt

SEQ ID NO: 4 - DNA, 10 bp, Artificial Sequence
  endonculease recognisiton sequence
  misc_feature (6)..(10): n is a, c, g, or t
gagtcnnnnn

SEQ ID NO: 5 - DNA, 10 bp, Artificial Sequence
  endonuclease recognition sequence
  misc_feature (6)..(10): n is a, c, g, or t
ctcagnnnnn

SEQ ID NO: 6 - DNA, 17 bp, Artificial Sequence
  oligo
agggtaaaccaaatacc

SEQ ID NO: 7 - DNA, 17 bp, Artificial Sequence
  oligo
agggttaaaccaccgcc

SEQ ID NO: 8 - DNA, 17 bp, Artificial Sequence
  oligo
agggtccttaacttgcc

SEQ ID NO: 9 - DNA, 15 bp, Artificial Sequence
  oligo
ccgggattggttgat

SEQ ID NO: 10 - DNA, 44 bp, Artificial Sequence
  template sequence
ggtatttggtttaccctgtgagactctggtatttggtttaccct

SEQ ID NO: 11 - DNA, 30 bp, Artificial Sequence; identical to SEQ ID NO: 1
  binder DNA

SEQ ID NO: 12 - DNA, 19 bp, Artificial Sequence
  primer
ggagcacaaaaccagttga

SEQ ID NO: 13 - DNA, 44 bp, Artificial Sequence
  template sequence
ggaactaatcagaccctgtgagactctggaactaatcagaccct

SEQ ID NO: 14 - DNA, 17 bp, Artificial Sequence
  trigger sequence
agggtctgattagttcc

SEQ ID NO: 15 - DNA, 30 bp, Artificial Sequence
  Binder DNA
agggtctgattagttcctggtccccaaaat

SEQ ID NO: 16 - DNA, 30 bp, Artificial Sequence
  HPV binder sequence
agggttaaggggtcggtggaccggtcgatg

SEQ ID NO: 17 - DNA, 20 bp, Artificial Sequence
  HPV trigger sequence
agggttaaggggtcggtgga

SEQ ID NO: 18 - DNA, 49 bp, Artificial Sequence
  HPV template sequence
tccaccgaccccttaaccctgtgagactctccaccgaccccttaaccct

(19) United States
(12) Patent Application Publication
(10) Pub. No.: US 2024/0309437 A1
(43) Pub. Date: Sep. 19, 2024

(54) TARGET RNA DETECTION

(71) Applicant: The University of Birmingham, Birmingham (GB)

(72) Inventors: James Tucker, Birmingham (GB); Timothy Dafforn, Warwickshire (GB); Jake Carter, Birmingham (GB); Jean-Loius Henry Alan Duprey, Birmingham (GB); Lorea Orueta Iturbe, Birmingham (GB); Matthew Hicks, West Midlands (GB)

(21) Appl. No.: 18/038,964

(22) PCT Filed: Dec. 3, 2021
(86) PCT No.: PCT/EP2021/084172

§ 371 Date: May 25, 2023

(30) Foreign Application Priority Data
Mar. 12, 2020 (GB) 2019059.1

(51) Int. Cl. C12Q 1/6848; C12Q 1/70

(52) U.S. Cl. CPC C12Q 1/6848; C12Q 1/701

(57) ABSTRACT

The present disclosure relates to methods and products for use in detecting target RNA sequences in a sample, based on a process which nicks the DNA strand of a DNA/RNA duplex and amplifies a product thereof

### FIELD

The present disclosure relates to methods and products for use in detecting target RNA sequences in a sample.

### BACKGROUND

CoVID-19 was declared a global pandemic on Mar. 11, 2020, with more than 118,000 cases reported in 114 different countries. Within three months, the number of cases had risen to nearly 6.5 million cases in 188 countries. In order to reduce its spread throughout the population, an accurate and efficient virus testing strategy is imperative. A key part of this strategy is continuous assay development, with the aim of reducing assay times and increasing sample throughput. Given that the clinical symptoms of a SARS-COV-2 infection can resemble those of the common cold and influenza, unambiguous identification of the virus itself is crucial for an effective diagnosis. In the early stages of the disease, this can only really be achieved by detecting viral RNA.

The diagnostics industry responded rapidly in developing a range of detection platforms, with the CDC, Thermo Fisher, and Public Health England, among many others, releasing tests to manage this unprecedented crisis.¹ The most common assays used for SARS-COV-2 detection are qPCR-based that take more than 60 minutes per sample and typically involve a two-step process. First, reverse transcriptase is used to convert viral RNA to complementary DNA (cDNA), a process that can take up to 30 minutes.² Then a quantitative polymerase chain reaction (qPCR) is performed to amplify the cDNA which is detected using a fluorescent dye, a process that takes up to an hour.³⁻⁵ To reduce assay times, a plethora of new approaches to SARS-COV-2 RNA detection have appeared in the literature in the past few months. Arguably the most successful of these have applied isothermal approaches to DNA amplification, greatly accelerating amplification speeds and hence reducing assay times.^{6,7} The most common isothermal amplification system is Loop mediated isothermal AMPlification (LAMP). LAMP methods have been developed for SARS-COV-2 but they struggle to give an result in <10 minutes.⁷

Another technique, which is often used in diagnostic assays, is the exponential amplification reaction (EXPAR). EXPAR reactions are restricted to DNA as the nick endonucleases required for the reaction are only active on DNA duplexes. This means that to use EXPAR to detect RNA an RNA->DNA step is required. This is generally provided by a reverse transcriptase (RT) reaction. Aside from often requiring an RT step, EXPAR is more rapid than other similar techniques as it is optimised to require only very short single stranded sequences to be amplified. The time taken for EXPAR assays cannot generally be optimized further as the resulting short fragments would not have the binding energy at the desired temperature to form duplexes required to prime the polymerase. The rate can be increased by increasing temperature but this also means that longer sequences are required to produce duplexes. This means that methods to increase EXPARs speed, such as EXPAR based assays, which employ a RT step, are limited. Moreover, any additional reactions necessary for conducting an assay will increase the total assay time further.

### SUMMARY

The present disclosure is based on the identification and use of an enzyme which is capable of nicking the DNA strand of an RNA/DNA duplex and the use of the nicked DNA to generate an amplified product. In one teaching, the amplified product may be generated using an, Exponential Amplification Reaction (EXPAR) In accordance with the teaching herein the inventors have shown that they can accurately identify RNA from samples, such as, CoVID-19 patient samples in less than 10 minutes, such as within 5 minutes.

In a first teaching, there is provided a method of detecting a target RNA sequence in a sample, the method comprising:
a) contacting the sample, at a first temperature, with a binder DNA molecule and a restriction endonuclease, which is capable of nicking the DNA strand of an RNA/DNA complex at a specific recognition sequence, in order to generate a nicked DNA molecule,
wherein the binder DNA molecule is capable of specifically binding to the target RNA sequence at the first temperature, thereby forming an RNA/DNA duplex: the binder DNA molecule comprising a portion of sequence, which does not bind to the target RNA sequence at the first temperature and which comprises a sequence is complementary to a template DNA molecule; and a further portion of sequence, which is capable of specifically binding to the target RNA sequence at the first temperature and which includes the specific recognition sequence,
wherein the restriction endonuclease nicks the bound binder DNA molecule at the recognition sequence, such that the nicked DNA molecule is capable of being released from the RNA/DNA duplex; or
a′) contacting the sample, at a first temperature, with a primer, a reverse transcriptase and a restriction endonuclease, which is capable of nicking the DNA strand of an RNA/DNA complex at a specific recognition sequence, in order to generate a nicked DNA molecule, wherein the primer is capable of specifically binding to the target RNA sequence at the first temperature, thereby forming an RNA/DNA duplex and initiating chain elongation and generation of an extended RNA/DNA duplex in the presence of dNTPs and the reverse transcriptase, the extended RNA/DNA duplex comprising the specific recognition sequence,
wherein the restriction endonuclease nicks the extended RNA/DNA duplex at the recognition sequence, such that the nicked DNA molecule is capable of being released from the RNA/DNA duplex; and
b) at a second temperature, contacting the nicked DNA molecule with the template DNA molecule, such that at least a 3′ portion of the nicked DNA molecule binds the template DNA molecule and initiates chain elongation and generation of an amplified product in the presence of dNTPs and a DNA polymerase;
   and detecting any of said amplified product, wherein detection of said amplified product, is indicative of the sample comprising the target RNA sequence to be detected.

As will be described in more detail herein, steps a) and b) may be carried out in a single receptacle/container, or two separate receptacles/containers.

In some teachings, the first and second temperature are the same temperature.

The target RNA sequence may be any suitable RNA sequence, including human RNA sequence and may include, mRNA, rRNA, siRNA, hnRNA, piRNA, aRNA, miRNA, RNA from an infectious agent and synthetic RNA molecules and the like. In one teaching, the target RNA sequence is from an infectious agent and the methods described herein may be employed in order to ascertain if the sample contains the infectious agent, or RNA from the infectious agent. The methods may therefore be used, in some teachings, to detect if a subject is infected with the infectious agent, or if a substrate is contaminated with the infectious agent, for example.

Moreover, for some integrating viruses (viruses which integrate into a host's genome), such as human papillomavirus (HPV), it may be better to detect the mRNA which transcribed, rather than detecting the viral DNA. This is because, in the case of HPV, expression of E6/E7 oncogenes of high-risk HPVs is necessary for the development and maintenance of a dysplastic phenotype. Thus, detection of E6/E7 mRNA may provide a better prognostic evaluation than simply detecting HPV DNA. Thus, the mRNA of HPV and other integrating viruses, which are transcribed in cells, may be detected in accordance with the present disclosure. Examples include Epstein-Barr virus (EBV); hepatitis, such as hepatitis B virus (HBV) and hepatitis C virus (HCV); adeno-associated virus-2 (AAV-2); and retroviruses, such as HIV and endogenous retrovirus (ERV1)

The infectious agent may be a bacteria, fungus or virus, for example. In certain teachings, the infectious agent is a single-stranded or double stranded RNA virus, or genome integrating virus, in which one or more genes of which is/are transcribed by a host cell. The teaching is particularly suited to the detection of single-stranded RNA viruses, including positive and negative stranded RNA viruses, for example.

Positive-strand RNA viruses are divided between the phyla Kitrinoviricota, Lenarviricota, and Pisuviricota (specifically classes Pisoniviricetes and Stelpavirictes) all of which are in the kingdom Orthornavirae and realm Riboviria. They are monophyletic and descended from a common RNA virus ancestor.

Positive-sense RNA viruses account for a large fraction of known viruses, including many pathogens such as the Hepatitis C virus, West Nile virus, dengue virus, and the SARS, MERS, and SARS-COV-2 coronaviruses, as well as less clinically serious pathogens such as the rhinoviruses that cause the common cold.

Exemplary positive strand RNA viruses include:
1. Bymoviruses, comoviruses, nepoviruses, nodaviruses, picornaviruses, potyviruses, sobemoviruses and a subset of luteoviruses (beet western yellows virus and potato leafroll virus)—the picorna like group (Picornavirata).
2. Carmoviruses, dianthoviruses, flaviviruses, pestiviruses, statoviruses, tombusviruses, single-stranded RNA bacteriophages, hepatitis C virus and a subset of luteoviruses (barley yellow dwarf virus)—the flavi like group (Flavivirata).
3. Alphaviruses, carlaviruses, furoviruses, hordeiviruses, potexviruses, rubiviruses, tobraviruses, tricornaviruses, tymoviruses, apple chlorotic leaf spot virus, beet yellows virus and hepatitis E virus—the alpha like group (Rubivirata).

Exemplary negative stranded RNA viruses include: Hepatitis D and Negarnaviricorta viruses, which include Haploviricotina and Polyploviricotina viruses. Notable Haploviricortina viruses include Ebola, Marburg, Measles, Mumps, RSV and rabies, for example. Notable Polyviricotina viruses include lassa and Influenza virus.

The present invention may be exemplified herein in terms of detecting Coronavirus and CoVID-19 in particular, but this should not be construed as limiting and the skilled addressee will easily appreciate and be able to adapt the present teaching to the detection of other RNA targets.

Any type of sample that may comprise a target RNA sequence may be used in the methods or with the kits disclosed herein. As such, the sample containing or suspected of containing a target RNA sequence is not specifically limited, and includes, for example, biological samples derived from living subjects, such as whole blood, serum, buffy coat, urine, faeces, cerebrospinal fluid, seminal fluid, saliva, tissue (such as lung tissue), cell cultures (such as mammalian cell cultures or bacterial cultures); samples containing RNA, such as viroids, viruses, bacteria, fungi, yeast, plants, and animals; samples (such as food and biological preparations) that may contain or be infected with microorganisms such as viruses or bacteria; and samples that may contain biological substances, such as soil, industrial process and manufacturing equipment, and wastewater; and samples derived from various water sources (e.g., drinking water). The sample may also be a swab of a solid surface, for example, suspected of containing or being contaminated by an infectious agent, for example.

Furthermore, a sample may be processed by any known method to prepare an RNA-containing composition used in the methods disclosed herein. Examples of such preparations can include cell breakage (e.g., cell lysates and extracts) and sample fractionation. A sample can be a single sample, or may be a plurality of samples (such as 2, 3, 4, 5, 6, 8, 10 or more samples), which are pooled together and tested in a single test.

As further described herein, the present inventors have identified that certain DNA restriction endonucleases are capable of recognising DNA/RNA duplexes, even though their normal/conventional target is a DNA/DNA duplex, and are capable of nicking the DNA strand of the DNA/RNA duplex. The binder DNA and/or primer molecules of the present invention must be able to specifically bind the target RNA sequence at the first temperature and the restriction endonuclease be capable of nicking the DNA strand of the DNA/RNA duplex at the recognition sequence. The binder DNA/primer molecule is, or comprises a portion of sequence, which is, complementary to the target RNA sequence, such that the binder DNA/primer is able to bind the target RNA at the first temperature. The binder DNA/primer is however, not able to bind the template DNA at the first temperature, or at least is not able to bind the template DNA and initiate chain elongation therefrom.

In a further aspect there is provided a binder DNA molecule for specifically binding an RNA template, the binder DNA molecule comprising:
a portion which is complementary to a target RNA molecule and which portion includes a DNA restriction endonuclease recognition site which may be nicked by a restriction endonuclease when the binder DNA is part of a DNA/RNA duplex; and a further portion which is not complementary and does not bind to the target RNA molecule, when the portion which is complementary to a target RNA molecule is bound to the target RNA molecule at a first temperature.

As mentioned herein, in one teaching, the present disclosure is directed to the detection of CoVID-19 and in this regard, the present teaching provides binder DNA sequences which are specifically designed for use in accordance with the present teaching, for detecting CoVID-19.

Thus, in one teaching for use in the detection of CoVID-19, the binder DNA molecule may consist essentially of, or consists of the sequence:

5′-AGG GTA AAC CAA ATA CCT GGT GTA TAC GTT-3′,
5′-AGG GTT AAA CCA CCG CCT GGA GAT CAA TTT-3′,
or
5′-AGG GTC CTT AAC TTG CCT GGT TGT GAT GGT-3′.

In one teaching, the binder DNA molecule consists essentially of, or consists of the sequence:

5′-AGG GTA AAC CAA ATA CCT GGT GTA TAC GTT-3′,

In this context, consists essentially of is intended to mean that one or more of the identified nucleotides, such as the 3′ nucleotide, may be chemically modified, providing that this does not substantially affect the ability of sequence binding to its target RNA sequence.

In one teaching, the binder DNA molecule comprises a portion, which binds the target RNA sequence at the first temperature, and a further portion, which does not bind the target RNA at the first temperature. The portion, which binds the target RNA sequence also includes the recognition sequence for the DNA restriction endonuclease which is capable of recognising DNA/RNA duplexes and nicking the DNA strand at its recognition sequence. The first temperature is chosen to limit or prevent any non-specific binding of the binder DNA/primer molecule to non-target RNA sequences and hence ensure that the binder DNA/primer only binds its specific target. In this regard, it may be appreciated that the portion, which binds the target RNA sequence may in some instances be fully complementary in terms of binding to its target RNA sequence. However, in some instances the portion of the binder DNA/primer which binds to the target RNA, may not be fully complementary and a number of miss-matches may be tolerated (such as 1, 2, 3 or 4 miss-matches, across the length of the portion of the binder DNA/primer sequence, which is capable of binding the target RNA at the first temperature). Such miss-matches may be in a region, which includes the recognition sequence of the DNA restriction endonuclease, in a region, which does not include the recognition sequence of the DNA restriction endonuclease, or both regions. In one teaching, any miss-matches may only be found in the region, which does not include the recognition sequence.

The binder DNA/primer molecule may be typically, 20-50 nucleotides in length, such as 25-40 nucleotides. When present, the region, which does not bind the target RNA sequence may be, for example, 2-8 nucleotides, such as 3-6 nucleotides in length. The region, which does not bind the target RNA molecule may be at the 5′ end of the binder DNA. However, the length of the binder DNA molecule and the target RNA binding and non-binding regions thereof, may also be determined in a functional manner. In this regard, the binder DNA must be capable of binding to its target RNA sequence at the first temperature, with the nicked DNA molecule derived therefrom, being released from the target RNA molecule at the first temperature. Thus, the binder DNA/primer molecule binds to the target RNA molecule at the first temperature, whereas the DNA nicked molecule does not.

The binder DNA may comprise chemical modifications such as are generally known in the art. In some embodiments, for example, the binder DNA can comprise chemically modified nucleotides (e.g., 2′-0 methyl derivative, phosphorothioates, etc.), 3′ end modifications, 5′ end modifications, or any combinations thereof. In some embodiments, the 3′ end of the binder DNA may be modified such that an extension reaction does not or cannot occur from the 3′ end of the binder DNA (e.g., upon binding to the target RNA sequence, or another non-target sequence, that might serve as a primer for polymerase extension). Any replication initiated from the 3′ end of the binder DNA may lead to detection errors (e.g., false positives). Accordingly, in some teachings, the binder DNA comprises a 3′ end modification that can reduce or eliminate the occurrence of any non-desired extension reactions, such as those discussed above. Non-limiting examples of 3′-end modifications include the addition of TAMRA, DABCYL, and FAM. Other non-limiting examples of modifications include, for example, biotinylation, fluorochromation, phosphorylation, thiolation, amination, or modified/inverted nucleotides, which do not readily permit the addition of a further nucleotide to the 3′ end of the binder DNA molecule under conventional DNA extension reactions using a DNA polymerase.

Using techniques known in the art, (see Ref 8, for example) the skilled addressee is able to calculate the melting temperature of any particular DNA sequence for a corresponding target RNA sequence, as well as the melting temperature of a nicked DNA sequence. The first temperature is chosen to be a temperature at which the binder DNA is expected to bind, as the first temperature is below the melting temperature of the binder DNA to the target RNA, but also being a temperature at which the restriction endonuclease and where appropriate, the reverse transcriptase, can function. Too high a temperature and the restriction endonuclease/reverse transcriptase may not function (e.g., the enzyme(s) may become denatured), too low a temperature and the nicking reaction and/or chain extension reaction may become too slow.

In the teaching where the binding DNA comprises a target RNA binding portion and a portion, which does not bind the target RNA, the first temperature will be chosen to be below the melting temperature of the binder DNA to the target RNA sequence, but above the melting temperature of the nicked DNA molecule, such that upon nicking, the nicked DNA molecule is released from the target RNA molecule. The skilled reader is readily able to take account of the effect on melting temperature, of any miss-matches across the length of the binder DNA, as well as the region, which does not bind the target RNA molecule and set a first temperature accordingly, using common general knowledge of the skilled reader.

Through appropriate design of the binder DNA molecule, taking into account its length, and miss-matches, the position of the recognition sequence and the length of the region which does not bind the target RNA molecule, the binder DNA molecule may be designed in order that its melting temperature for its target RNA molecule is/may be in the range of 45-70° C. and the melting temperature of the nicked DNA molecule is/may be in the range of 30-45° C. The first temperature is chosen to be below the melting temperature of the binder DNA molecule, but above the melting temperature of the nicked DNA molecule. Thus, for example, if the melting temperature of a binder DNA molecule to a complementary nucleic acid molecule is calculated as 60° C. and the melting temperature of the nicked DNA molecule is calculated as 35° C., the first temperature may be between 45° C.-55° C., for example, in order to ensure the binder DNA molecule binds the target RNA molecule and the nicked DNA does not and is released from the target RNA molecule.

The nicked DNA sequence comprises the region of sequence, which does not bind (and hence is non-complementary) to the target RNA sequence, as well as a region which is complementary with the RNA target and does bind to the RNA target prior to the nicking reaction taking place.

According to the methods described herein, which utilise a primer and a reverse transcriptase, the primer may be complementary to its target RNA sequence and hence not include a portion which is intended not to bind its target RNA sequence. However, unlike the teaching relating to the use of a binder DNA which comprises target RNA binding and non-binding regions and which does not utilise a reverse transcriptase, the nicked DNA sequence may not be released from the target RNA sequence simply by the first temperature being above the melting temperature of the nicked DNA molecule to its target RNA sequence. In such an instance, the reverse transcriptase and/or other processing enzyme may facilitate in releasing the nicked DNA molecule from the target RNA sequence.

As described above, the length of the nicked single stranded DNA molecule can depend on the design of the binder DNA molecule. However, typically the nicked single stranded DNA molecule may be 10-30 nucleotides in length, such as 12-24 nucleotides, or 14-20 nucleotides in length, or any integers or ranges between the upper and lower values, such as 10-20, or 14-30 nucleotides in length.

A generalised method of designing a binder DNA according to the present disclosure, may involve first looking for all BstNI, AvrII or PhoI restriction sites in a DNA sequence, which would be complementary to a potential target RNA. Once a suitable target region has been identified to which a binder DNA molecule with such a restriction site would bind, carrying out one or more (such as at least two, three, or four) of the following steps:
1) From any potential binder DNA sequences, eliminating the sequences with multiple BstNI, AvrII or PhoI restriction sites (such as two, three, four or more restriction sites) and sequences which comprise ploy A's;
2) Designing binder DNA sequences with AGGG at the 5′ end;
3) If more than one potential binder DNA sequence is identified, determining the Tm's of the potential binder DNA sequences and removing the sequences with the highest and lowest Tm's
4) Assessing the 3′ end of the nicked DNA/Trigger sequence that would be produced following nicking of the binder DNA molecule, to see if the bases CCAC were present. The sequence with the most matches of CCAC are selected in addition to binder DNA sequences, which possessed the highest and lowest Tm following step 3.

The DNA restriction endonuclease may be any restriction endonuclease, which is capable of nicking or preferentially nicking the DNA strand of a DNA/RNA duplex. By “preferentially nicking”, we mean that in a DNA/RNA duplex, the DNA strand is nicked to a greater extent that the RNA strand. By greater extent, we mean greater than 60%, 70%, 80%, 90% or 95%, with respect to the RNA strand. One such DNA restriction endonuclease is BstNI. Murray et al Nucleic Acids Research, 2010, Vol. 38, No. 22 8257-8268 showed that some restriction endonucleases can nick RNA:DNA heteroduplexes. For example BstNI (cut site CC/WGG where W=A or T), efficiently cleaves only the DNA strand of an RNA/DNA heteroduplex. This means that enzymes like BstNI can be used to cleave a DNA/RNA heteroduplex to yield a nicked DNA molecule, in accordance with the present teaching. Crucially a restriction endonuclease is not processive so requires only a single reaction to yield the fragment. Reverse transcriptase (RT), for example, would be required to catalyse the formation of a number of phosphodiester bonds before a DNA fragment was generated for an amplification reaction. This makes an endonuclease reaction, such as BstNI reaction, inherently faster than the RT reaction. Other restriction enzymes which are capable of cleaving the DNA strand of an RNA/DNA duplex include AvrII, NdeI and PhoI. See for example: http://rebase.neb.com/cgi-bin/hybridlist and Ref 14.

Following release of the nicked DNA molecule, from the target RNA molecule, the nicked DNA molecule binds to/with a longer template DNA molecule and is able to initiate chain elongation and generation of an amplified product in the presence of dNTPs and a DNA polymerase. The template molecule is designed such that the nicked DNA molecule is capable of specifically binding to it and initiate chain elongation. In one teaching, the amplified product is generated through an EXPAR technique known in the art.⁹⁻¹¹

Exemplary amplification reactions may rapidly synthesize short oligonucleotides (e.g., 8-16 bases) specified by the sequence within the template DNA molecule. Versions of the reactions known to the skilled addressee can proceed in either a linear or an exponential amplification reaction (EXPAR). Another example is Rolling Circle Amplification (RCA) and Loop-mediated isothermal amplification (LAMP). Both of these reactions utilize simple, stable isothermal (i.e., non-thermocycling) conditions. Thermocycling reactions, such as polymerase chain reaction (PCR) and versions thereof may also be employed. The rate of amplification depends entirely on the molecular parameters governing the interactions of the molecules in the reaction. The exponential version of the method is a molecular chain reaction that may use the oligonucleotide products of each linear reaction to create more of the same nicked DNA molecule for use in subsequent rounds of amplification and generation of amplified product.

The nicked DNA molecule may bind to a 3′ end or end region of the template DNA and amplify a 5′ end or end region of the template DNA molecule. The template DNA molecule may typically be less than 100 nucleotides, such as less than 70, 60 or 50 nucleotides in length, and any integers in between. The template DNA molecule is longer than the nicked DNA molecule and may be at least double the length of the nicked shortened single stranded DNA molecule, in some instances, as will be described in more detail herein.

At least in the one receptacle/container embodiment, where steps a) and b) are conducted in a single receptacle/container, such as an Eppendorf tube, well of a multi-well plate, or the like, the melting temperature of the nicked DNA molecule with the template DNA molecule will be higher than for the target RNA molecule. Thus, the second temperature can be chosen such that the nicked DNA molecule will be capable of binding the template DNA molecule and be capable of initiating chain elongation, but not capable of binding the target RNA molecule. In one teaching, the first and second temperatures are the same, but this need not always be the case. For example, if the first temperature was 55° C. and the melting temperature of the nicked DNA molecule to the target DNA molecule was between 30° C.-45° C., the melting temperature of the nicked DNA molecule with the template DNA molecule may be between 55° C.-65° C., for example. That being the case, the second temperature may be a temperature, which is lower than the calculated melting temperature of the nicked DNA with the template DNA, for example (such as 40° C.-50° C.).

In one embodiment, the generation of an amplified product is carried out by an isothermal reaction for amplifying DNA, such as LAMP or EXPAR. Isothermal amplification methods provide detection of a nucleic acid target sequence in a streamlined, exponential manner, and are not limited by the constraint of thermal cycling, such as when employing polymerase chain reaction (PCR). Many isothermal techniques are known to the skilled addressee, but they all share some features in common. For example, because the DNA strands are not heat denatured, all isothermal methods rely on an alternative approach to enable primer binding and initiation of the amplification reaction: a polymerase with strand-displacement activity. Once the reaction is initiated, the polymerase must also separate the strand that is still annealed to the sequence of interest.

Isothermal methods typically employ unique DNA polymerases for separating duplex DNA. DNA polymerases with this ability include Klenow exo-, Bsu large fragment, and phi29 for moderate temperature reactions (25-40° C.) and the large fragment of Bst DNA polymerase for higher temperature (50-65° C., such as 50° C., 55° C., or 60° C.) reactions. Methods according to the present teaching may be performed under isothermal or substantially constant temperature conditions. In embodiments that relate to performing the method under a substantially constant temperature, some fluctuation in temperature is permitted. For example, in some embodiments a substantially constant temperature may fluctuate within a desired or identified target temperature range (e.g., about +/−2° C. or about +/−5° C.). In embodiments, a substantially constant temperature may include temperatures that do not include thermal cycling, such as employed when conducting PCR. In some embodiments, the second temperature at or below about the calculated/predicted or experimentally determined optimal hybridization or melting temperature of the template DNA molecule, such as 5° C.-10° C. or below about the calculated/predicted or experimentally determined optimal hybridization or melting temperature of the template DNA molecule. In some embodiments, for convenience, the first and second temperatures are the same or substantially the same (e.g., about +/−2° C. or about +/−5° C.).

In one embodiment the present teaching utilises strand displacement amplification (SDA) or nicking enzyme amplification reaction (NEAR). Both techniques rely on a strand-displacing DNA polymerase, typically Bst DNA Polymerase, Bsu Large Fragment or Klenow Fragment from E. coli, to initiate at nicks created by a strand-limited restriction endonuclease or nicking enzyme at a site contained in the template DNA molecule. The nicking site is regenerated with each polymerase displacement step, resulting in exponential amplification. NEAR is extremely rapid and sensitive, enabling detection of small target amounts in minutes.

One such suitable nicking enzyme is Nt. BstNBI. Nt. BstNBI is a site specific endonuclease that cleaves only one strand of DNA on a double-stranded DNA substrate. The nicking endonuclease catalyzes a single strand break 4 bases beyond the 3′ side of the recognition sequence. Other non-limiting examples of suitable nicking enzymes include Nb.BbvCI, Nt.AlwI, Nt.BbvCI, Nb.BsrDI, Nb.BtsI, Nt. BspQI, Nb.BsmI, Nt.CviPII, and Nt. BsmAI.

The methods described herein may be carried out in any suitable format, including in single receptacles, such as Eppendorf or other tubes, or in a multiple format such as multiwell plates, for example. Additionally cartridge formats, such as lateral flow or microfluidic based cartridges, for use with or without an associated reader, may be envisaged.

The amplified product may be detected by techniques known in the art, for example luminescence, UV or visible spectroscopy or spectrometry, fluorescence spectroscopy or spectrometry, mass spectrometry, liquid chromatography, fluorescence polarization, electrochemistry, electrophoresis, enzyme labeling (such as peroxidase or alkaline phosphatase), fluorescent labeling (such as fluorescein or rhodamine), chemiluminescence, bioluminescence, surface plasmon resonance (SPR), or a fluorophore-modified probe DNA (e.g., TaqMan probe), visually by naked eye, by way of a suitable chromogenic/colour change or generation (such as by using a suitable tag or dye-modified probe or nucleic acid) or a combination of the above technologies.

Typically, the total time from contacting the binder DNA with the target RNA sequence, to detecting sufficient (such as, the detectable signal which is greater than or equal to 5, 6, 7, 8, 9 or 10 standard deviations from a baseline value—typically obtained at the start of an assay, before any signal generation occurs) amplified product may be less than 15 minutes, less than 10 minutes, less than 9, 8, 7, 6, or even 5 minutes.

The key to the speed of EXPAR is twofold; firstly, the amplification occurs at one temperature, thus avoiding lengthy heating and cooling steps, and secondly the amplified sequence is relatively small (typically 15-20 bases long). These two factors result in EXPAR, once triggered, producing ca. 10⁹ strands of DNA product in a matter of minutes.^{9,12} In accordance with the present teaching a nicked single stranded DNA molecule (the trigger) starts the EXPAR reaction by interacting with a template DNA molecule. Large quantities of short double stranded DNA sequences are then generated in an isothermal cycle involving a DNA polymerase to extend the sequence and a nicking endonuclease to cut it, while leaving the template intact (see Scheme 1a). As with the qPCR assay, duplex formation may be monitored spectroscopically, for example through the use of a fluorescent dye, e.g. SYBR Green II.¹³

The binder DNA and template DNA molecules as described herein can be made synthetically and may be synthesized using a phosphoramidite method, a phosphotriester method, an H-phosphonate method, or a thiophosphonate method known in the art. After synthesis, the binder DNA and template DNA molecules can be purified for example using ion exchange HPLC.

The terms “3” and “5” are used herein to describe the location of a particular site within a single strand of nucleic acid. When a location in a nucleic acid is “3′ to” or “3′ of a reference nucleotide or a reference nucleotide sequence, this means that the location is between the 3′ terminus of the reference nucleotide or the reference nucleotide sequence and the 3′ hydroxyl of that strand of the nucleic acid. Likewise, when a location in a nucleic acid is “5′ to” or “5′ of” a reference nucleotide or a reference nucleotide sequence, this means that it is between the 5′ terminus of the reference nucleotide or the reference nucleotide sequence and the 5′ phosphate of that strand of the nucleic acid.

As used herein, “nicking” refers to the cleavage of only one strand of a fully double-stranded nucleic acid molecule or a double-stranded portion of a partially double-stranded nucleic acid molecule at a specific position relative to a nucleotide sequence that is recognized by the enzyme that performs the nicking. The specific position where the nucleic acid is nicked is referred to as the “nicking site” (NS).

For example, the recognition sequence and the nicking site of an exemplary double-stranded DNA nicking endonuclease, N.BstNB I, are shown below with “▾” to indicate the cleavage site and N to indicate any nucleotide:

▾
5′-G A G T C N N N N N-3′
3′-C T C A G N N N N N-5′

The sequence of the sense strand of the Nt.BstNBI recognition sequence is 5′-GAGTC-3′, whereas that of the antisense strand is 5′-GACTC-3′.

“Initiates chain elongation”, refers to the process by which the nicked DNA molecule acts as a primer, when bound to the template DNA and addition of a nucleotide to the 3′ end of the nicked DNA molecule, which is complementary to the next base present on the template DNA. In this manner, the length of the nicked DNA grows or elongates with each addition of a further nucleotide to the extended 3′ end of the nicked DNA molecule. This also applies to the teaching where a primer binds to a target RNA sequence and reverse transcriptase is employed in order to generate a reverse complement DNA sequence of the target RNA.

The term “generation of an amplified product” refers to the process of making more than one copy of a template DNA molecule, or portion thereof, using a nicked DNA molecule as a primer that comprises a sequence complementary to the 5′ end or region of the template DNA.

As used herein, the phrase “amplified product” refers to a product which comprises one or more copies of a particular nucleic acid molecule. Typically, the amplified product comprises at least 103, 104, 105, 106, 107, 108, 109, or more copies of the nucleic acid molecule.

In a further aspect there is provided a kit comprising, consisting essentially of, or consisting of:
a binder DNA molecule as defined herein; and
a restriction endonuclease which is capable of nicking the binder DNA sequence when bound to a target RNA molecule, at the recognition sequence of the restriction endonuclease.

In one teaching for use in the detection of CoVID-19, the binder DNA molecule consists essentially of, or consists of the sequence:

5′-AGG GTA AAC CAA ATA CCT GGT GTA TAC GTT-3′,
5′-AGG GTT AAA CCA CCG CCT GGA GAT CAA TTT-3′,
or
5′-AGG GTC CTT AAC TTG CCT GGT TGT GAT GGT-3′.

In one teaching, the binder DNA molecule consists essentially of, or consists of the sequence:

5′-AGG GTA AAC CAA ATA CCT GGT GTA TAC GTT-3′,

In this context, consists essentially of is intended to mean that one or more of the identified nucleotides, such as the 3′ nucleotide, may be chemically modified, providing that this does not substantially affect the ability of sequence binding to its target RNA sequence.

In one teaching the restriction endonuclease is BstNI.

The kit may optionally further include:
a template DNA molecule to which the nicked DNA molecule can bind and initiate chain elongation, in order to generate an amplified product, in the presence of (optionally tagged or dye-labelled) dNTPs and a DNA polymerase. The kit may further include (optionally tagged or dye-labelled) dNTPs and/or a DNA polymerase, such as Bst DNA Polymerase, Large Fragment or Klenow Fragment (3′-5′ exo-) and optionally a DNA nicking enzyme such as Nt. BstNBI.

In some embodiments the methods and/or kits may further comprise additional reagents. Some non-limiting examples of other reagents that can be used in accordance with the teaching herein include metallic salts such as sodium chloride, magnesium chloride, magnesium acetate, and magnesium sulfate; substrates such as dNTP mix; and buffer solutions such as Tris-HCl buffer, tricine buffer, sodium phosphate buffer, and potassium phosphate buffer. Likewise, detergents, oxidants and reducing agents can also be used in the practice of the methods disclosed herein and/or a DNA intercalating dye, such as SYBR Green I, or a tag or dye labelled probe which is designed to bind to any amplified product, may be further be provided.

Embodiments of the disclosure will now be described by way of example, and with reference to the accompanying figures in which:

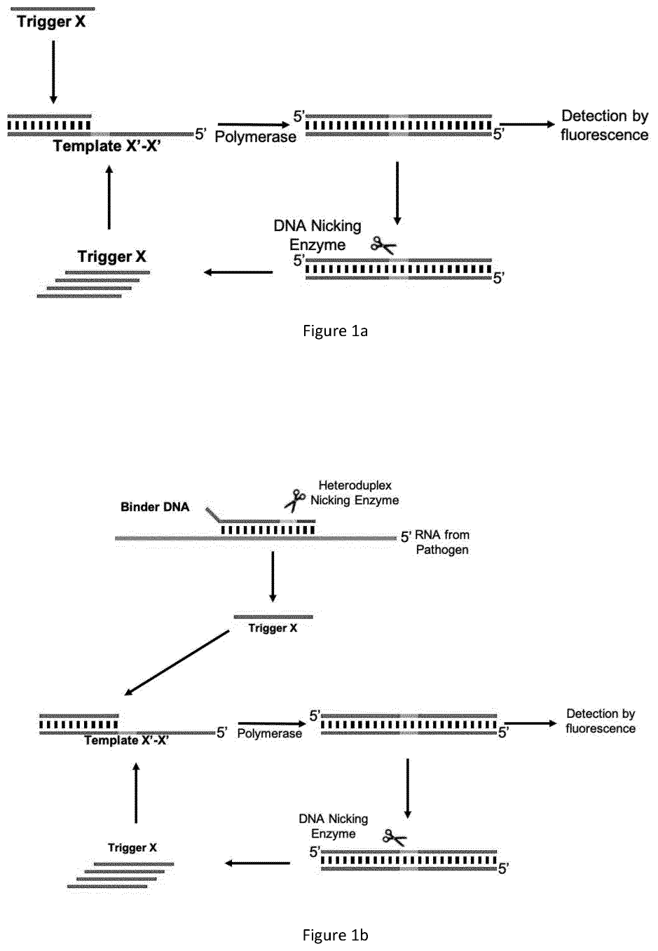

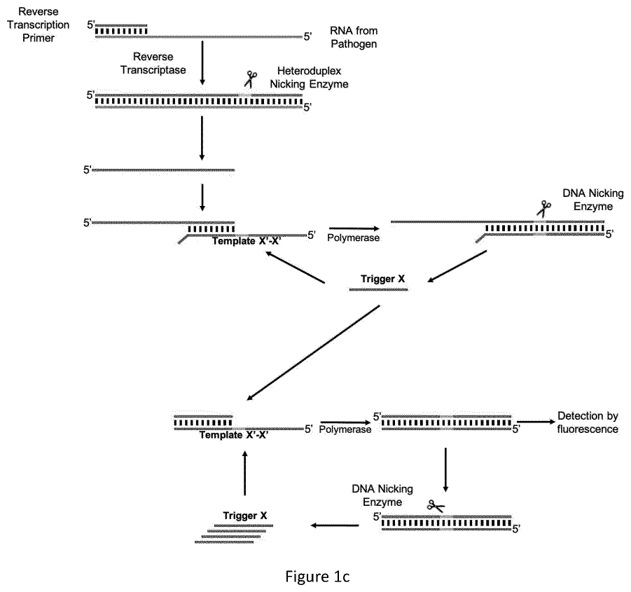

FIG. 1 shows schematically (a) The Exponential Amplification Reaction. Trigger X anneals to Template X′-X′ and is extended by a DNA polymerase (Bst 2.0 polymerase). The top strand of the now duplex DNA is then cut by a nicking enzyme (Nt. BstNBI). The released DNA is identical to Trigger X and is therefore capable of priming another Template X′-X′. (b) Reverse Transcriptase-Free EXPAR. Binder DNA anneals to viral RNA. The DNA of the DNA:RNA heteroduplex is nicked by the restriction endonuclease BstNI that as a nicking enzyme by only cutting DNA. The released DNA strand is Trigger X and is amplified by EXPAR; (c) A reverse transcriptase primer anneals to viral RNA and is converted into cDNA by reverse transcriptase (SuperScript IV Reverse Transcriptase). The RNA:DNA heteroduplex formation creates a recognition site for a restriction endonuclease (BstNI) to cleave the DNA strand. The cleaved cDNA strand contains the Trigger X sequence, which primes Template X′-X′ and is extended to form the full duplex. The top strand of the now duplex DNA is then cut by a nicking enzyme (Nt.BstNBI). The released DNA is Trigger X and is therefore capable of priming another Template X′-X′

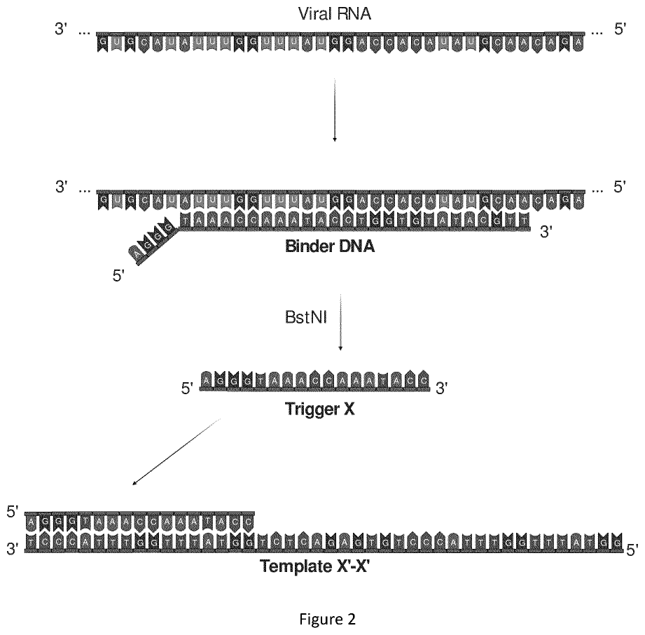

FIG. 2 shows in more detail to FIG. 1, an example with specific sequences of a CoVID-19 target RNA sequence, a binder DNA sequence which can bind the target RNA, a nicked DNA sequence and a template DNA sequence;

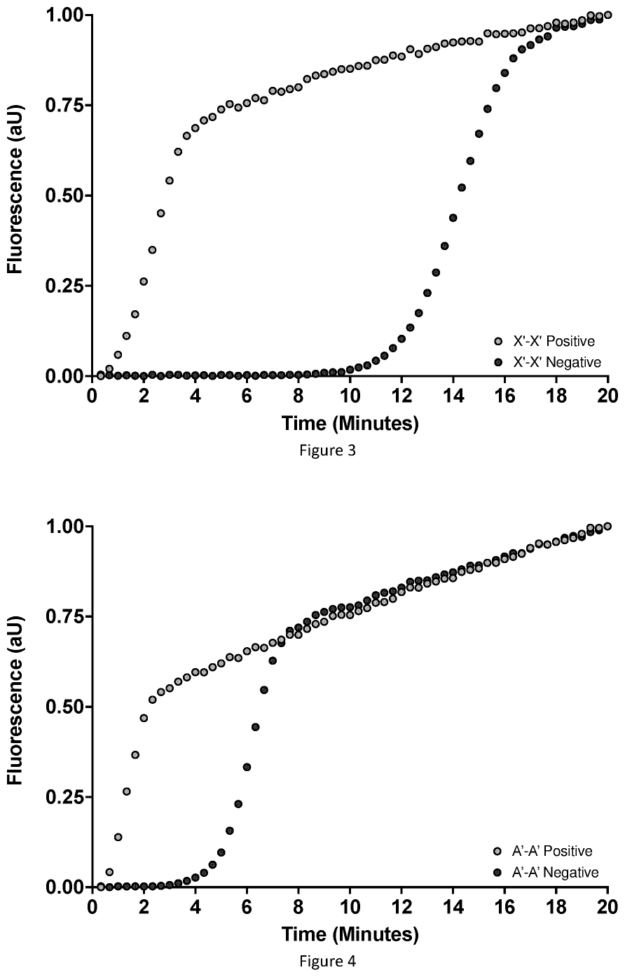

FIG. 3 is a plot showing the normalised fluorescence units against time following amplification Trigger X and a blank.

FIG. 4 is a plot showing the normalised fluorescence units against time following amplification Trigger A and a blank.

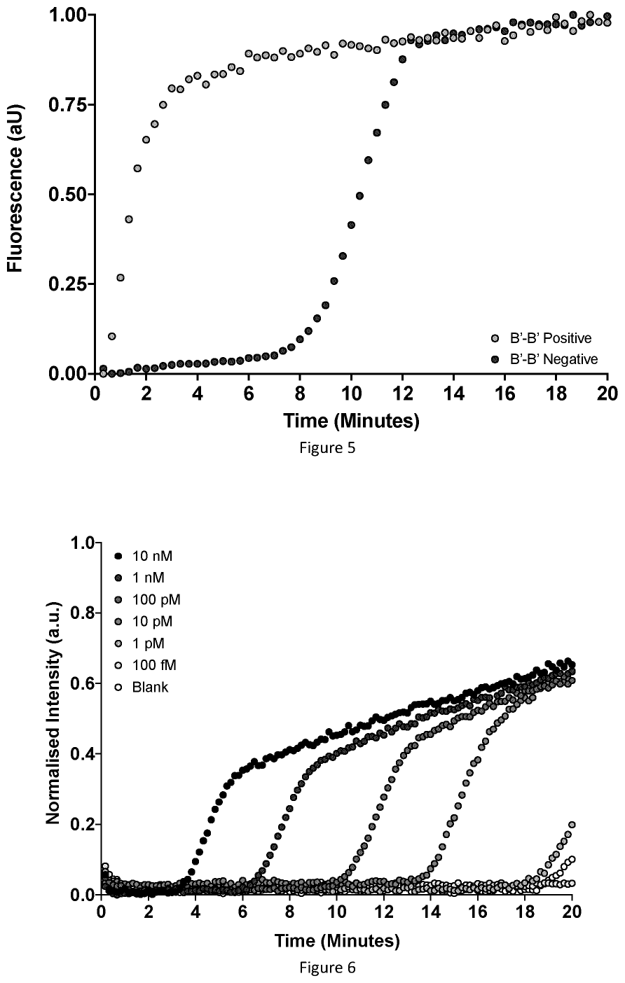

FIG. 5 is a plot showing the normalised fluorescence units against time following amplification Trigger B and a blank.

FIG. 6 is a plot showing the normalised fluorescence units against time following amplification Trigger X at concentrations ranging from 10 nM-100 fM and a blank.

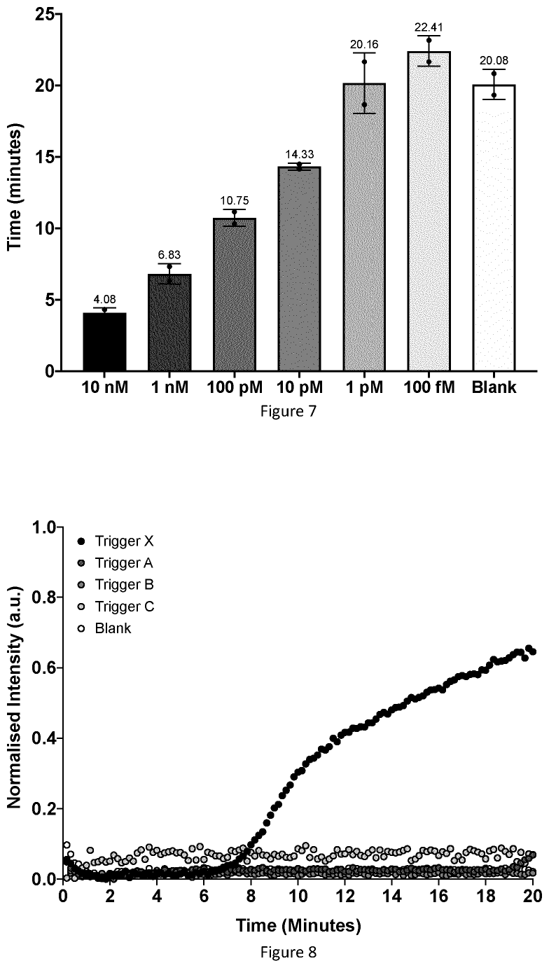

FIG. 7 is a bar chart showing the time where the fluorescence signal was greater than 10 standard deviations from the baseline, following amplification Trigger X at concentrations ranging from 10 nM-100 fM and a blank.

FIG. 8 is a plot showing the normalised fluorescence units against time following amplification of Trigger X, Trigger A, Trigger B and a blank using Template X′-X′.

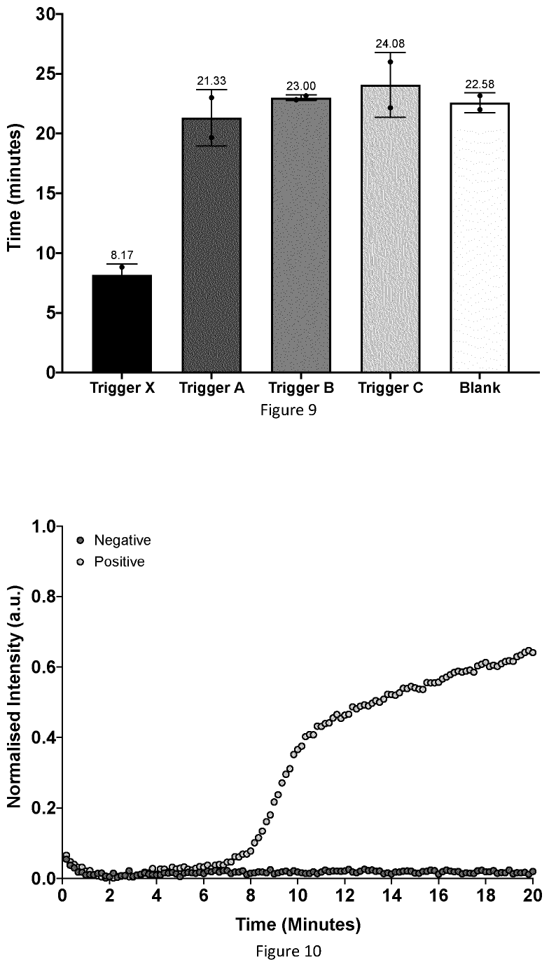

FIG. 9 is a bar chart showing the time where the fluorescence signal was greater than 10 standard deviations from the baseline, following amplification of Trigger X, Trigger A, Trigger B and a blank using Template X′-X′.

FIG. 10 is a plot showing the normalised fluorescence units against time following two pot reverse transcriptase EXPAR, of a patient positive and negative sample.

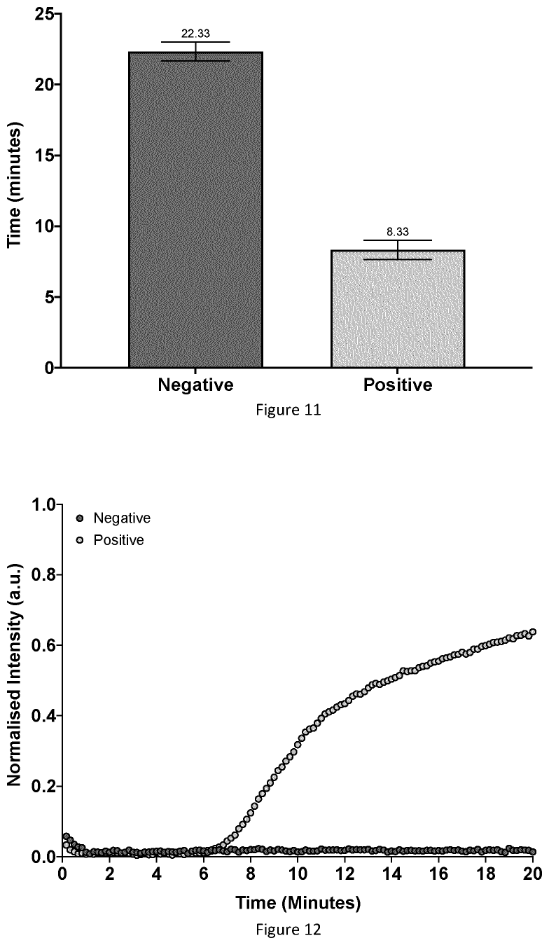

FIG. 11 is a bar chart showing the time where the fluorescence signal was greater than 10 standard deviations from the baseline, following two pot reverse transcriptase EXPAR, of a patient positive and negative sample.

FIG. 12 is a plot showing the normalised fluorescence units against time following one pot reverse transcriptase EXPAR, of a patient positive and negative sample.

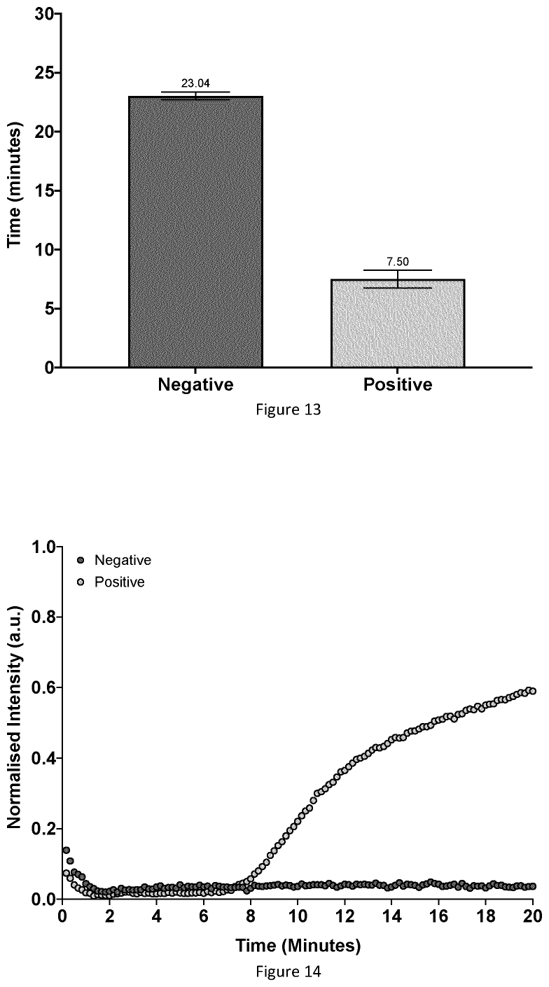

FIG. 13 is a bar chart showing the time where the fluorescence signal was greater than 10 standard deviations from the baseline, following one pot reverse transcriptase EXPAR, of a patient positive and negative sample.

FIG. 14 is a plot showing the normalised fluorescence units against time following two pot reverse transcriptase EXPAR, of a patient positive and negative sample.

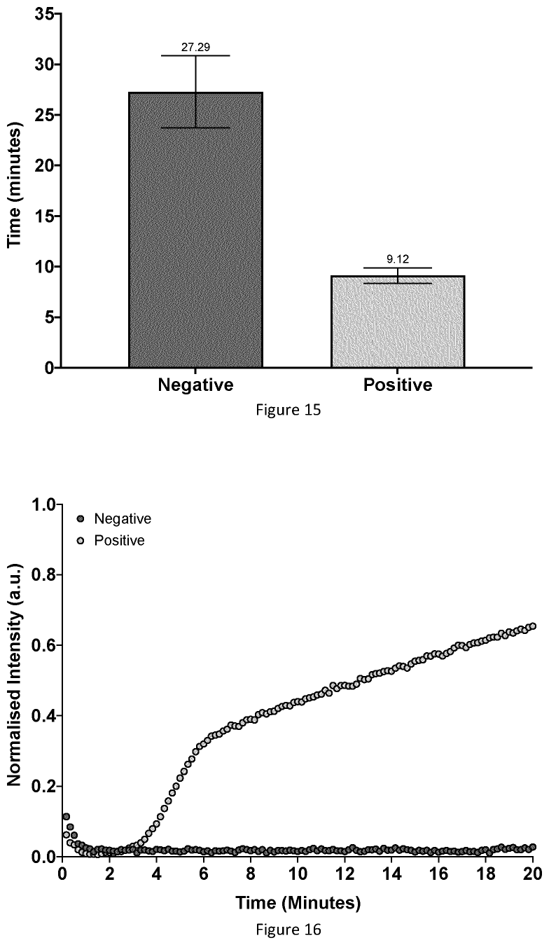

FIG. 15 is a bar chart showing the time where the fluorescence signal was greater than 10 standard deviations from the baseline, following two pot reverse transcriptase EXPAR, of a patient positive and negative sample.

FIG. 16 is a plot showing the normalised fluorescence units against time following one pot reverse transcriptase EXPAR, of a patient positive and negative sample.

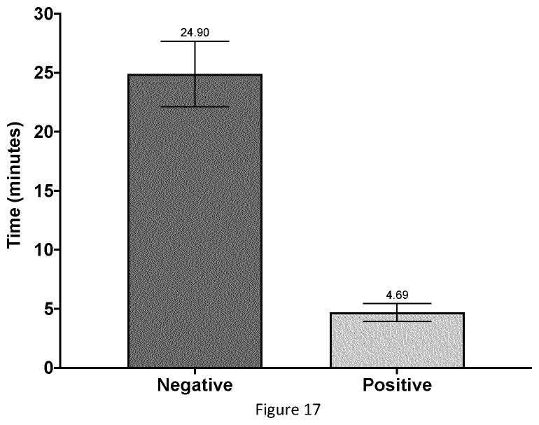

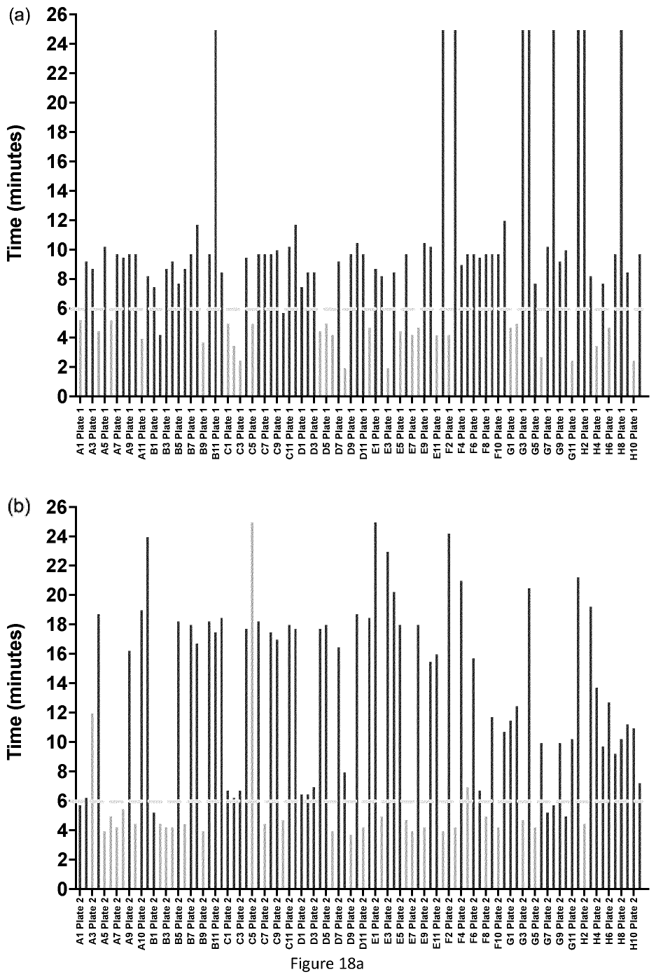

FIG. 17 is a bar chart showing the time where the fluorescence signal was greater than 10 standard deviations from the baseline, following one pot reverse transcriptase EXPAR, of a patient positive and negative sample.

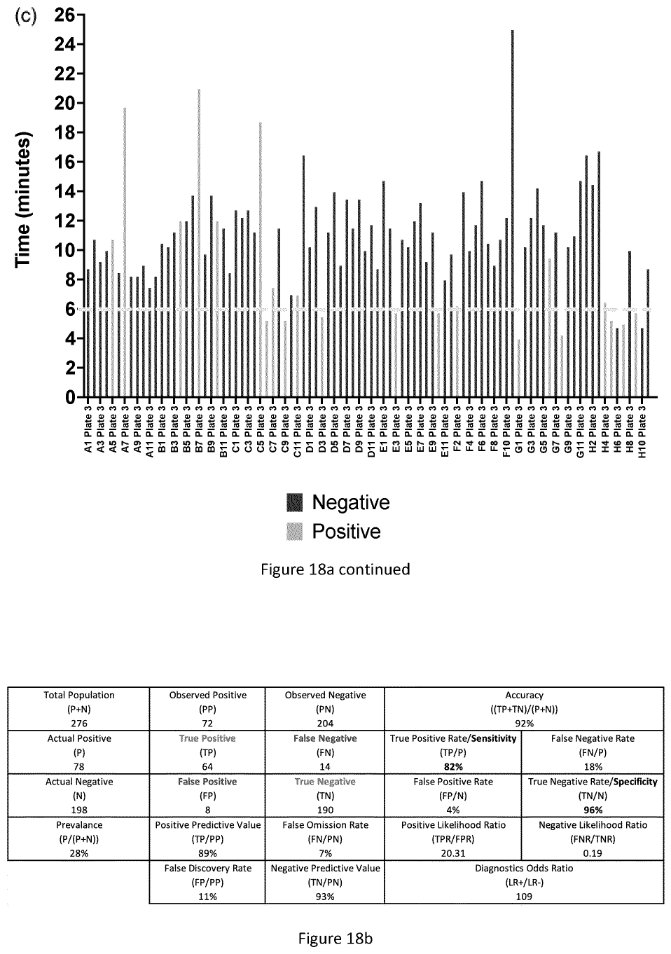

FIG. 18 shows results from patient samples looking at COVID19 Orf1ab gene a) RTF-EXPAR assay data for patient validation samples, showing the time for RTF-EXPAR to produce a signal. Red bars represent positive samples, blue bars represent negative samples, and the yellow dashed lines represent the cut off time of 6 minutes. Each run time was calculated to be the point at which the fluorescence signal was greater than 10 standard deviations from the baseline signal (10-sigma time); and b) Confusion matrix displaying the results of the patient sample validation study.

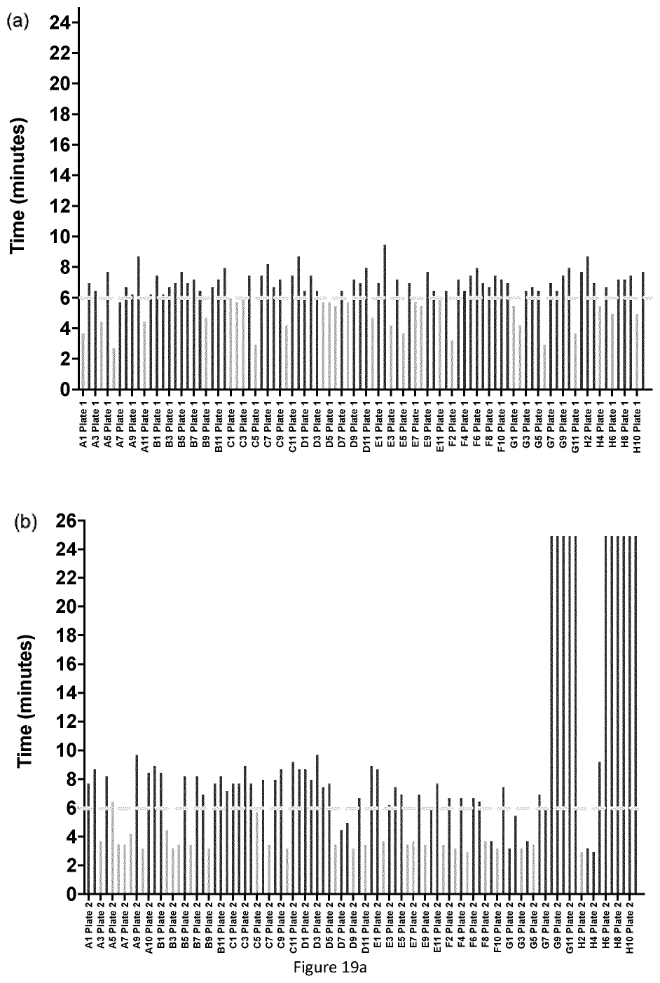

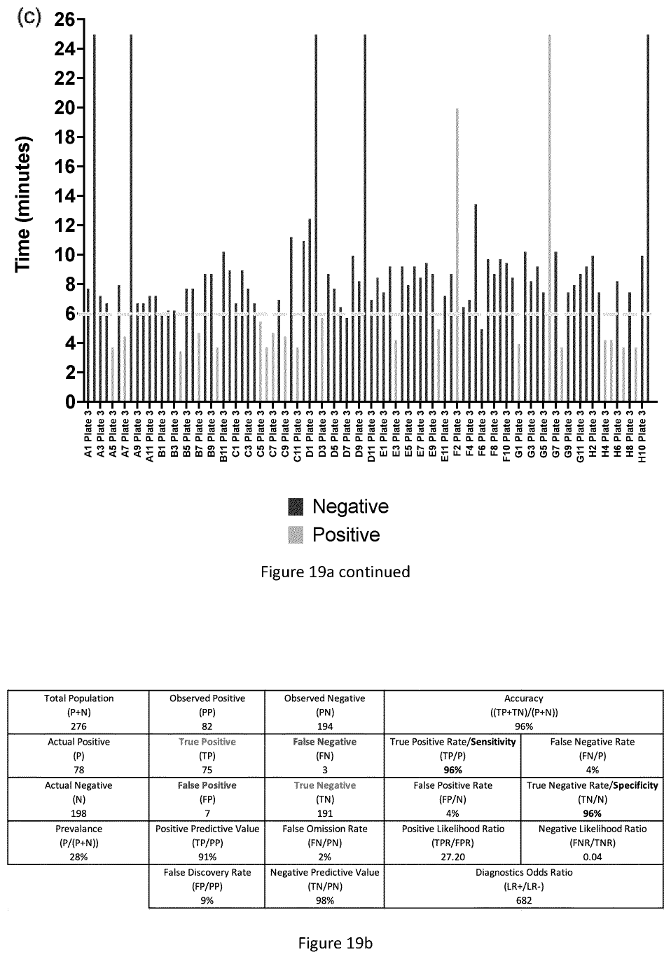

FIG. 19 shows results from patient samples looking at COVID19 N gene a) RTF-EXPAR assay data for patient validation samples, showing the time for RTF-EXPAR to produce a signal. Red bars represent positive samples, blue bars represent negative samples, and the yellow dashed lines represent the cut off time of 6 minutes. Each run time was calculated to be the point at which the fluorescence signal was greater than 10 standard deviations from the baseline signal (10-sigma time); and b) Confusion matrix displaying the results of the patient sample validation study.

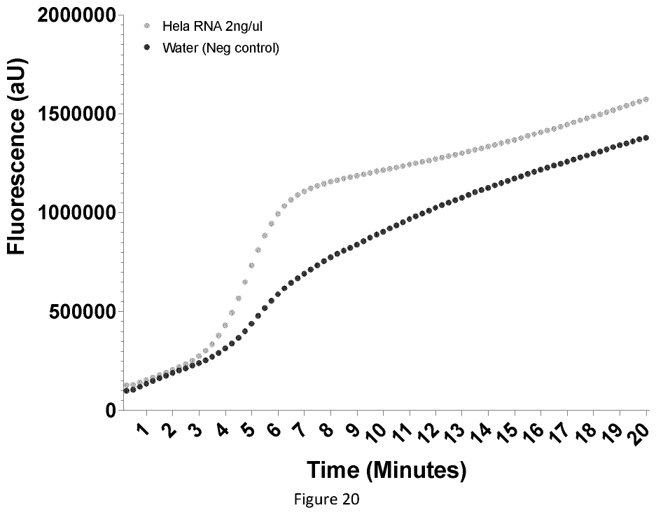

FIG. 20 is a plot showing the normalised fluorescence units against time following one pot reverse transcriptase EXPAR, of 2 ng/μL of HPV mRNA and a negative control.

### MATERIALS

Milli-Q water purified with a Millipore Elix-Gradient A10 system (resistivity >18 μΩ·cm, TOC≤5 ppb, Millipore, France) was used in all the experiments. Nt.BstNBI, BstNI and Bst 2.0 Polymerase were obtained from New England Biolabs (Hitchin, UK) as was the buffer, 10× Isothermal amplification buffer (200 mM Tris-HCl, 100 mM (NH₄)₂SO₄, 500 mM KCl, 20 mM MgSO₄, 1% Tween 20, pH 8.8) which was used in all the experiments. Superscript IV Reverse Transcriptase was obtained from ThermoFisher (Paisley, UK), DMSO (>=99%) was obtained from Fisher Scientific (Loughborough, UK) and dsGreen 100× (an analogue of SYBR Green I), was obtained from Lumiprobe (Hannover, De). Bovine Serum Albumin (BSA, diluted to 4 mg/mL in water) and Single-Stranded Binding Protein (SSB, solution of 0.5 mgs in 20 mM Tris-HCl, pH 8.0, 0.5 M NaCl, 0.1 mM EDTA, 0.1 mM DTT, 50% Glycerol) was obtained from Sigma-Aldrich (Dorset, UK). All the nucleotide triphosphates and oligonucleotide sequences (desalted) were obtained from Sigma-Aldrich (Dorset, UK). SARS-COV-2 RNA and cDNA patient samples (in MagNA Pure elution buffer) were obtained from Public Health England (PHE) and stored at −80° C.

### Phe Samples:

All clinical specimens were handled in a Containment Level 2 laboratory. To prepare each sample, Viral Transfer Medium (VTM, 300 μL, Medical Wire ViroCult) from a nose and throat swab was added to Buffer AL (Qíagen) in a 1:1 ratio and heated to 60° C. for 30 minutes in a calibrated heat block. Samples were then extracted on the MagNAPure96 (Roche) automated extraction system and then run on the Abbott M2000 RT-qPCR Test for SARS-COV-2 RNA Detection. For EXPAR assay development, positive and negative samples from the SARS-COV-2 RNA assays were separately combined in MagNA Pure elution buffer (giving 29,080 RNA copies/μL for the combined positive sample). Upon receipt from PHE, each sample was diluted 400-fold, aliquoted into 50 μL vials and stored at −80° C. Prior to use, each sample was submerged in ice and allowed to slowly melt; once melted the sample was used immediately before being cooled for storage again at −80° C.

TABLE 1
Oligonucleotides used for the during EXPAR.

| Name | Sequence (5′- 3′) |
|---|---|
| Trigger X | AGG GTA AAC CAA ATA CC |
| Trigger A | AGG GTT AAA CCA CCG CC |
| Trigger B | AGG GTC CTT AAC TTG CC |
| Trigger C | CCG GGA TTG GTT GAT |
| Template X′-X′ | GGT ATT TGG TTT ACC CTG TGA GAC TCT GGT ATT TGG TTT ACC |
| | CT |
| Binder DNA | 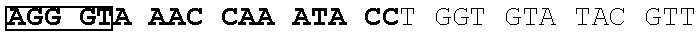 |
| RT Primer | GGA GCA CAA AAC CAG TTG A |
| Template N′-N′ | GGA ACT AAT CAG ACC CTG TGA GAC TCT GGA ACT AAT CAG ACC |
| | CT |
| Trigger N | AGG GTC TGA TTA GTT CC |
| Binder DNA N | 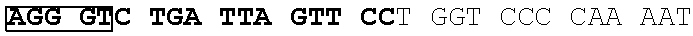 |

Key: 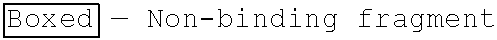 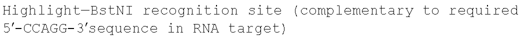
Bold-Trigger X sequence

### Data Analysis and Classification

To analyse the EXPAR real-time fluorescence amplification curves we developed a program in C# which performs analysis of fluorescence data. The program analyses the first 20 data points and calculates the mean value and standard deviation as a base line. Following generation of these two values, each subsequent data point is analysed to determine if its value minus the average value is greater than 10 standard deviations away from the mean. The cycle which meets this criterion is converted into a time and used as the minimum amplification time. Should this time be less than 20 minutes the output is TRUE indicating the presence of SARS-COV-2 RNA, times after 20 will generate a FALSE output indicating the absence of SARS-COV-2 RNA.

### Example

A key element to developing a successful EXPAR assay is the identification of optimal nucleotide sequences in the target genome.

Qian et al previously found that the particular trigger sequence used in EXPAR plays a vital role in determining its success.¹² Adapting their approach we designed a 17-mer DNA trigger for EXPAR (Trigger X, FIG. 1a) to be complementary to a sequence of Orf1ab¹⁴ in the SARS-COV-2 genome.

The design of the Binder DNA sequence had to be designed such that it was able to:
1) Have enough base pairing interactions to produce a stable complex with the relevant sequence on the RNA
2) generate a sequence that could be recognized and nicked by the relevant enzyme
3) contain a non-complementary sequence (such as at the 5′ end) that would allow it to anneal to the DNA Template X′-X′ sequence for the next stage of the reaction

To achieve this we designed a Binder DNA sequence to include a “tail” at the 5′ end that is not complementary to viral RNA yet is complementary to the Template X′-X′ sequence. The length and sequence of the “tail” had to be designed so that it did not form any secondary structure that would inhibit binding to the RNA or that would trigger the EXPAR reaction in the absence of RNA. The sequence of the “Tail” sequence also had to provide enough binding energy for the binding to the Template X′-X′ to ensure a duplex was formed that was stable enough to allow polymerase catalysed elongation of a strand complementary to the Template X′-X′ (see FIG. 1b).

The sequence of the tail also need to be such that the EXPAR reaction could proceed once it was released from the DNA:RNA heteroduplex. Exemplary tail sequences were developed in accordance with the teaching of Qian et al. However, briefly the process involves the following steps:
1) All BstNI restriction sites in the region of interest were found with 10 bases at the 5′ and 3′ end.
2) From these the sequences with multiple restriction sites and ploy A's were eliminated
3) From these remaining sequences AGGG was added at the 5′ end (as these bases have been shown to be important for EXPAR (Qian et al))
4) The Tm's of these Binder sequences were analysed and the sequences with the highest and lowest Tm's removed
5) The sequences we selected were those which possessed a high and low Tm and those which possessed the most bases CCAC at the 3′ end. (Qian et al teachings).

In more detail, FIG. 2 identifies a specific CoVID-19 viral target RNA sequence (a) and the binder DNA sequence, which was designed to bind this target RNA sequence (b). As shown, the binder DNA comprises a 3′ portion which is complementary to the target RNA sequence and which includes a BstNI recognition sequence (CCTGG), as well as a non-complementary 5′ end. Upon nicking of the binder DNA by BstNI, the nicked DNA (labelled Trigger X) is released from the target RNA. The nicked DNA binds to the template DNA (labelled Template X′-X′) and is able to initiate chain elongation and generation of an amplified DNA/DNA duplex template X′-X′. The sequence of the template DNA is designed to have repeating sequences at either end of the template, with a spacer in the middle, which has a recognition sequence for Nt.BstNBI. Action of Nt. BstNBI nicks the top strand of the DNA/DNA duplex and the polymerase molecule is able to continue to elongate from the nicked site, displacing a copy of the trigger X molecule which can bind a template X′-X′ molecule and initiate another round of amplification. In this manner an exponential generation of an amplified DNA/DNA duplex template X′-X′ molecule can take place and generation of the increasing numbers of such DNA/DNA duplex molecules can be detected by, for example the use of a dye which is capable of binding DNA/DNA duplex molecules.

In FIG. 1c, a specific CoVID-19 RNA target sequence is identified, a primer sequence anneals and is extended using a reverse transcriptase. Elongation over a BstNI recognition sequence (CCTGG) allows the DNA strand to be cleaved and displaced by strand displacement activity of the transcriptase enzyme. The cleaved DNA strand is complementary to Template X′-X′ which is extended by Bst polymerase to form an DNA/DNA duplex of template X′-X′. The recognition sequence for Nt.BstNBI enables the top strand of the DNA/DNA duplex to be nicked, displacing a copy of the trigger X molecule which can bind a template X′-X′ molecule and initiate EXPAR.

### Example 1: Determining which Trigger Sequence is Most Suitable

This experiment tests the three triggers designed for this process against their respective template sequence, in order to determine the sequence which provides the largest separation between specific and unspecific amplification.

### Methods

### Part A:

1.50 μL of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

5.55 μL of water, 5.00 μL of 10× Isothermal amplification buffer, 1.50 μL of Template (1 μM), 2.40 μL of MgSO₄ (100 mM), 1.50 μL dNTP (10 nM), 0.75 μL of dsGreen (1:5 dilution in DMSO from 100× to 20×), 0.30 μL of SSB solution.

### Part C:

3 μL of one trigger at 100 nM (Trigger X or Trigger A or Trigger B)

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 55° C. The fluorescence is measured every 10 seconds over an incubation time of at least 20 minutes.

### Results

The results of the experiment are shown in FIGS. 3-5. The x axis of the plots shows the time. All the tests show amplification of samples containing Trigger within 2 minutes. The time until unspecific amplification varies with Trigger, with Trigger X taking more than 9 minutes for the signal to increase. Based on the fluorescence amplification of the unspecific samples Trigger X was chosen as the most successful trigger and carried forward.

### Example 2: Determining Sensitivity of the EXPAR Sequence

Optimisation steps were carried out using Trigger X and Template X′-X′ which resulted in a larger separation between positive and negative amplifications. The template concentration was decreased from 50 nM to 25 nM and the reaction temperature was decreased from 55° C. to 50° C. Using this modified procedure, the sensitivity of the EXPAR reaction was observed using Trigger X over a range of concentrations (10 nM-100 fM).

### Methods

### Part A:

1.50 ML of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

6.30 μL of water, 5.00 μL of 10× Isothermal amplification buffer, 0.75 μL of Template X′-X′ (1 μM), 2.40 μL of MgSO₄ (100 mM), 1.50 μL dNTP (10 nM), 0.75 μL of dsGreen (1:5 dilution in DMSO from 100× to 20×), 0.30 μL of SSB solution.

### Part C:

3 μL of Trigger X at (100 nM, 10 nM, 1 nM, 100 pM, 10 pM, 1 pM, Blank).

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 50° C. The fluorescence is measured every 10 seconds over an incubation time of at least 20 minutes.

### Results

The results are shown in FIGS. 6-7. As the concentration of Trigger X decreases the time for amplification to occur increases. These results indicate that a higher concentration of Trigger X will produce amplification faster, therefore a higher concentration of Binder DNA is more preferable.

### Example 3: Determining the Specificity of the EXPAR Procedure

This experiment tests multiple non-specific trigger sequence against Template X′-X′ sequence, in order to determine if any sequence can produce amplification.

### Methods

### Part A:

1.50 ML of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

6.30 μL of water, 5.00 μL of 10× Isothermal amplification buffer, 0.75 μL of Template X′-X′ (1 μM), 2.40 μL of MgSO₄ (100 mM), 1.50 μL dNTP (10 nM), 0.75 μL of dsGreen (1:5 dilution in DMSO from 100× to 20×), 0.30 μL of SSB solution.

### Part C:

3 μL of one trigger sequence at (Trigger X, Trigger A, Trigger B and Trigger C)

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 50° C. The fluorescence is measured every 10 seconds over an incubation time of at least 20 minutes.

### Results

The results are shown in FIGS. 8-9. These data sets show that the fully complementary Trigger X is able to produce amplification after 8 minutes. On the contrary the non-specific triggers are unable to produce amplification until after 20 minutes. This indicates the reaction is highly sequence specific.

### Example 4: Two Pot Reverse Transcription EXPAR

This experiment tests if the two pot rt-EXPAR procedure is capable of detecting the presence of SARS-COV-2 cDNA following production from viral RNA. By using a patient positive and negative sample, it was assessed if there would be a differentiation in amplification times.

### Methods:

### Part A:

1.50 μL of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

6.30 μL of water, 5.00 μL of 10× Isothermal amplification buffer, 0.75 μL of Template X′-X′ (1 μM), 2.40 μL of MgSO₄ (100 mM), 1.50 μL dNTP (10 nM), 0.75 μL of dsGreen (1/5 dilution in DMSO), 0.30 μL of SSB solution.

### Part C:

2 μL BstNI (10 U/μL) and 3 μL of viral cDNA.

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 50° C. The fluorescence is measured every 10 seconds over an incubation time of at least 20 minutes.

### Results:

The results are shown in FIGS. 10-11. These data sets show that the patient positive sample produced amplification after 8.33 minutes, whereas the patient negative sample produces amplification after 22.33 minutes. This indicates that there is a significant difference between the amplification times of the samples and that detection of cDNA is possible.

### Example 5: One Pot Reverse Transcription EXPAR

This experiment tests if the one pot rt-EXPAR procedure is capable of detecting the presence of SARS-COV-2 RNA. By using a patient positive and negative sample, it was assessed if there would be a differentiation in amplification times.

### Methods:

### Part A:

1.50 μL of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

6.30 μL of water, 5.00 μL of 10× Isothermal amplification buffer, 0.75 μL of Template X′-X′ (1 M), 2.40 μL of MgSO₄ (100 mM), 1.50 μL dNTP (10 nM), 0.75 μL of dsGreen (1/5 dilution in DMSO), 0.30 μL of SSB solution.

### Part C:

1 μL of rt-Primer (100 nM), 1 μL of BstNI (10 U/μL), 3 μL of viral RNA (72.7 copies/μL) and 0.1 μL of SuperScript IV Reserve Transcriptase (200 U/μL),

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 50° C. The fluorescence is measured every 10 seconds over an incubation time of at least 20 minutes.

### Results:

The results are shown in FIGS. 12-13. These data sets show that the patient positive sample produced amplification after 7.50 minutes, whereas the patient negative sample produces amplification after 23.04 minutes. This indicates that there is a significant difference between the amplification times of the samples and that detection of RNA is possible.

### Example 6: Two Pot Reverse Transcription Free EXPAR

This experiment tests if the two pot rtf-EXPAR procedure is capable of detecting the presence of SARS-COV-2 RNA using the binder DNA sequence and the enzyme BstNI. By using a patient positive and negative sample, it was assessed if there would be a differentiation in amplification times.

### Methods:

### Part A:

1.50 ML of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

6.30 μL of water, 5.00 μL of 10× Isothermal amplification buffer, 0.75 μL of Template X′-X′ (1 μM), 2.40 μL of MgSO₄ (100 mM), 1.50 μL dNTP (10 nM), 0.75 μL of dsGreen (1:5 dilution in DMSO from 100× to 20×), 0.30 μL of SSB solution.

### Part C:

10 μL of RNA:DNA heteroduplex digestion mixture, prepared as follows: 25 μL of water, 5 μL of 10× Isothermal amplification buffer, 5 μL BstNI (10 U/μL), 10 μL of Binder DNA (1 μM) and 5 μL of viral RNA (72.7 copies/μL). The mixture is then incubated at 50° C. for 5 minutes.

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 50° C. The fluorescence is measured every 10 seconds over an incubation time of at least 20 minutes

### Results:

The results are shown in FIGS. 14-15. These data sets show that the patient positive sample produced amplification after 9.12 minutes, whereas the patient negative sample produces amplification after 27.29 minutes. This indicates that there is a significant difference between the amplification times of the samples and that detection of RNA is possible through the use of Binder DNA.

### Example 7: One Pot Reverse Transcription Free EXPAR

This experiment tests if the one pot rtf-EXPAR procedure is capable of detecting the presence of SARS-COV-2 RNA using the binder DNA sequence and the enzyme BstNI in a single pot. By using a patient positive and negative sample, it was assessed if there would be a differentiation in amplification times.

### Methods:

### Part A:

1.50 ML of water, 2.50 μL of 10× Isothermal amplification buffer, 3.75 μL of BSA solution, 1.50 μL of Bst 2.0 DNA polymerase (1.6 U/μL), 0.75 μL of Nt. BstNBI (10 U/μL).

### Part B:

6.30 μL of water, 5.00 μL of 10× Isothermal amplification buffer, 0.75 μL of Template X′-X′ (1 μM), 2.40 μL of MgSO₄ (100 mM), 1.50 μL dNTP (10 nM), 0.75 μL of dsGreen (1:5 dilution in DMSO from 100× to 20×), 0.30 μL of SSB solution.

### Part C:

1 μL BstNI (10 U/μL), 2 μL of Binder DNA (1 μM) and 3 μL of viral RNA (72.7 copies/μL).

### Addition Step:

Part B (17 μL) is added to a PCR tube cooled at 4° C. To this, Part C is added and kept at 4° C. for 5 mins, before addition of Part A (10 μL). The tube is then sealed, with the contents then subjected to amplification.

### Amplification Step:

Isothermal incubation and fluorescence signal measurements are performed using an Agilent Mx3005P Real-Time PCR system (Didcot, UK) set to a constant temperature of 50° C. The fluorescence is measured every 10 seconds over an incubation time of at least 20 minutes

### Results:

The results are shown in FIGS. 16-17. These data sets show that the patient positive sample produced amplification after 4.69 minutes, whereas the patient negative sample produces amplification after 24.90 minutes. This indicates that there is a significant difference between the amplification times of the samples and that detection of RNA is possible through the use of Binder DNA.

In conclusion, through the use of a new reverse transcriptase-free EXPAR method involving a DNA-selective restriction endonuclease, we have demonstrated the successful detection of SARS-COV-2 RNA in less than ten minutes, with an amplification time of less than five minutes. These speeds not only are much faster than qPCR (assay time of at least 60 minutes) but also currently outperform LAMP and 30-minute lateral flow tests. This rtf-EXPAR method would be completely compatible (deployment ready) for use on equipment currently used for qPCR-based CoVID-19 assays. The simplicity and speed of the assay enables this method to be modified to detect a range of infectious diseases as described hereinabove, such as influenza, respiratory syncytial virus and Ebola to name but a few.

All references referred to herein, are hereby incorporated herein in their entirety.

### Example 8: Patient Validation Studies for Detecting COVI19 on the Basis of the Orf1ab and N Genes

### Exponential Amplification Reaction

The protocol first involves the preparation of three solutions, Part A, Part B and Part C, followed by an addition step and then finally an amplification step.

### Part A

1.50 μL of water, 2.50 μL of 10× Isothermal amplification buffer, 3.75 μL of BSA solution, 1.50 μL of Bst 2.0 DNA polymerase (1.6 U/μL) and then 0.75 μL of Nt.BstNBI (10 U/μL).

### Part B

6.30 μL of water, 5.00 μL of 10× Isothermal amplification buffer, 0.75 μL of Template X′-X′ (1 UM), 2.40 μL of MgSO₄ (100 mM), 1.50 μL dNTP (10 nM), 0.75 μL of dsGreen (1:5 dilution in DMSO from 100× to 20×) and then 0.30 μL of SSB solution.

### Part C

RTF EXPAR assay (one-pot RTF-EXPAR): Reagents are mixed together as follows: 1 μL BstNI (2 U/μL), 2 μL of Binder DNA X (100 nM), and then 3 μL of positive or negative sample.

### Addition Step

Part B (17 μL) is added to a PCR tube, and to this is added Part C (6 μL), followed by Part A (10 μL). The tube is then sealed, with the contents then subjected to amplification.

### EXPAR Amplification

Isothermal incubation and fluorescence signal measurements are performed using a Thermo Fisher QuantStudio 5 Real-Time PCR system, 96-well, 0.2 mL. The temperature is set at 25° C. for 15 seconds, before being raised to 50° C. for the duration of the assay, with the fluorescence reading measured every 10 seconds over an incubation time of 30 min.

### Results

The results are shown in FIGS. 18-19. These data sets show a cut off time of 6 minutes. All samples with an amplification time of less than 6 minutes was recorded as a positive whereas any time over 6 minutes was recorded as a negative. FIGS. 18a and b display the results of the Orf1ab gene target showing a sensitivity of 82% and an negativity of 96%. FIGS. 19a and b display the results of the N gene target showing a sensitivity of 96% and an negativity of 96%. This indicates that there is a significant difference between the amplification times of the samples with and without RNA.

Example 9;: HPV example

| Name | Sequence 5′-3′ |
|---|---|
| Binder DNA | AGG GTT AAG GGG TCG GTG GAC |
| HPV | CGG TCG ATG |
| Trigger HPV | AGG GTT AAG GGG TCG GTG GA |
| Template | TCC ACC GAC CCC TTA ACC CTG TGA GAC |
| HPV′-HPV′ | TCT CCA CCG ACC CCT TAA CCC T |

### Exponential Amplification Reaction

The protocol first involves the preparation of three solutions, Part A, Part B and Part C, followed by an addition step and then finally an amplification step.

### Part A

1.50 μL of water, 2.50 μL of 10× Isothermal amplification buffer, 3.75 μL of BSA solution, 1.50 μL of Bst 2.0 DNA polymerase (1.6 U/μL) and then 0.75 μL of Nt.BstNBI (10 U/μL).

### Part B

6.30 μL of water, 5.00 μL of 10× Isothermal amplification buffer, 0.75 μL of Template HPV′-HPV′ (1 μM), 2.40 μL of MgSO₄ (100 mM), 1.50 μL dNTP (10 nM), 0.75 UL of dsGreen (1:5 dilution in DMSO from 100× to 20×) and then 0.30 μL of SSB solution.

### Part C

RTF EXPAR assay (one-pot RTF-EXPAR): Reagents are mixed together as follows: 1 μL BstNI (2 U/μL), 2 μL of Binder DNA HPV (100 nM), and then 3 μL of positive or negative sample.

### Addition Step

Part B (17 μL) is added to a PCR tube, and to this is added Part C (6 μL), followed by Part A (10 μL). The tube is then sealed, with the contents then subjected to amplification.

### EXPAR Amplification

Isothermal incubation and fluorescence signal measurements are performed using a Thermo Fisher QuantStudio 5 Real-Time PCR system, 96-well, 0.2 mL. The temperature is set at 25° C. for 15 seconds, before being raised to 50° C. for the duration of the assay, with the fluorescence reading measured every 10 seconds over an incubation time of 30 min.

### Results

The results are shown in FIG. 20. These data sets show that the mRNA positive sample produced amplification after 4.5 minutes, whereas the negative sample produces amplification after 6 minutes. This indicates that there is a significant difference between the amplification times of the samples and that detection of mRNA is possible through the use of Binder DNA and the methods described herein.

### REFERENCES

1. Sheridan, C. Coronavirus and the race to distribute reliable diagnostics. Nature Biotechnology 38, 382-384 (2020).
2. Standard Reverse Transcription Protocol (Two-step). Sigma-Aldrich https://www.sigmaaldrich.com/technical-documents/protocols/biology/standard-reverse-transcription-protocol-two-step.html.
3. Notomi, T. Loop-mediated isothermal amplification of DNA. Nucleic Acids Research 28, 63e-663 (2000).
4. Ali, M. M. et al. Rolling circle amplification: a versatile tool for chemical biology, materials science and medicine. Chemical Society Reviews 43, 3324 (2014).
5. Hindson, B. J. et al. High-Throughput Droplet Digital PCR System for Absolute Quantitation of DNA Copy Number. Anal. Chem. 83, 8604-8610 (2011).
6. Asiello, P. J. & Baeumner, A. J. Miniaturized isothermal nucleic acid amplification, a review. Lab on a Chip 11, 1420 (2011).
7. Carter, L. J. et al. Assay Techniques and Test Development for COVID-19 Diagnosis. ACS Cent. Sci. 6, 591-605 (2020).
8. Oligo Analyzer. https://eu.idtdna.com/calc/analyzer.
9. Tan, E. et al. Isothermal DNA Amplification Coupled with DNA Nanosphere-Based Colorimetric Detection. Anal. Chem. 77, 7984-7992 (2005).
10. Reid, M. S., Le, X. C. & Zhang, H. Exponential Isothermal Amplification of Nucleic Acids and Assays for Proteins, Cells, Small Molecules, and Enzyme Activities: An EXPAR Example. Angewandte Chemie International Edition 57, 11856-11866 (2018).
11. Mok, E., Wee, E., Wang, Y. & Trau, M. Comprehensive evaluation of molecular enhancers of the isothermal exponential amplification reaction. Scientific Reports 6, (2016).
12. Qian, J. et al. Sequence dependence of isothermal DNA amplification via EXPAR. Nucleic Acids Research 40, e87-e87 (2012).
13. SYBR® Green Based Quantitative PCR. Sigma-Aldrich https://www.sigmaaldrich.com/life-science/molecular-biology/pcr/quantitative-pcr/sybr-green-based-qpcr.html.
14. Murray, I et al Sequence-specific cleavage of RNA by Type II restriction enzymes. Nucleic Acids Research 30(22) p 8257-8268 (2010)

1. A method of detecting a target RNA sequence in a sample, the method comprising:
a) contacting the sample, at a first temperature, with a binder DNA molecule and a restriction endonuclease, which is capable of nicking the DNA strand of an RNA/DNA complex at a specific recognition sequence, in order to generate a nicked DNA molecule,
wherein the binder DNA molecule is capable of specifically binding to the target RNA sequence at the first temperature, thereby forming an RNA/DNA duplex: the binder DNA molecule comprising a portion of sequence, which does not bind to the target RNA sequence at the first temperature and which comprises a sequence is complementary to a template DNA molecule; and a further portion of sequence, which is capable of specifically binding to the target RNA sequence at the first temperature and which includes the specific recognition sequence,
wherein the restriction endonuclease nicks the bound binder DNA molecule at the recognition sequence, such that the nicked DNA molecule is capable of being released from the RNA/DNA duplex; or
a′) contacting the sample, at a first temperature, with a binder DNA molecule, a reverse transcriptase and a restriction endonuclease, which is capable of nicking the DNA strand of an RNA/DNA complex at a specific recognition sequence, in order to generate a nicked DNA molecule,
wherein the binder DNA molecule is capable of specifically binding to the target RNA sequence at the first temperature, thereby forming an RNA/DNA duplex and initiating chain elongation and generation of an extended RNA/DNA duplex in the presence of dNTPs and the reverse transcriptase, the extended RNA/DNA duplex comprising the specific recognition sequence,
wherein the restriction endonuclease nicks the extended RNA/DNA duplex at the recognition sequence, such that the nicked DNA molecule is capable of being released from the RNA/DNA duplex; and
b) at a second temperature, contacting the nicked DNA molecule with the template DNA molecule, such that the nicked DNA molecule binds the template DNA molecule and initiates chain elongation and generation of an amplified product in the presence of dNTPs and a DNA polymerase;
and detecting any of said amplified product, wherein detection of said amplified product, is indicative of the sample comprising the target RNA sequence to be detected.

2. The method according to claim 1, wherein steps a)/a′) and b) are conducted in a single reaction vessel, receptacle or container.

3. The method according to either of claims 1 and 2 wherein the first and second temperatures are the same.

4. The method according to any preceding claim wherein target RNA sequence is an mRNA; rRNA; siRNA; hnRNA; piRNA; aRNA; miRNA; an RNA from an infectious agent; or a synthetic RNA molecule.

5. The method according to claim 4, wherein the infectious agent is a bacteria, fungus or virus.

6. The method according to claim 5, wherein the virus is a single-stranded or double stranded RNA virus, or a virus which integrates into a host cell's genome and is transcribed to generate mRNA.

7. The method according to claim 6 wherein the virus is a positive-sense RNA virus, such as, Hepatitis C virus, West Nile virus, dengue virus, SARS, MERS, and SARS-COV-2 coronavirus.

8. The method according to claim 7, wherein the SARS-COV-2 coronavirus is CoVID-19.

9. The method according to any preceding claim, wherein the binder DNA molecule of step a) has a melting temperature for its target RNA molecule in the range of 45-70° C. and a melting temperature of the nicked DNA molecule is in the range of 30-45° C.

10. The method according to any preceding claim, wherein the DNA restriction endonuclease is BstNI, AvrII or PhoI.

11. The method according to any preceding claim, wherein the amplified product is generated by a variable thermal cycling (such a PCR), or isothermal amplification technique and/or linear or an exponential amplification reaction (EXPAR).

12. The method according to claim 11, wherein the EXPAR includes a strand displacement amplification (SDA) or nicking enzyme amplification reaction (NEAR).

13. The method according to any preceding claim wherein the amplified product is detected by luminescence, UV or visible spectroscopy or spectrometry, fluorescence spectroscopy or spectrometry, mass spectrometry, liquid chromatography, fluorescence polarization, electrochemistry, electrophoresis, enzyme labelling, fluorescent labelling, chemiluminescence, bioluminescence, surface plasmon resonance (SPR), a fluorophore-modified probe DNA, visually by naked-eye, by way of a suitable chromogenic/colour change or generation (such as by using a suitable tag or dye-modified probe or nucleic acid) or any combination thereof.

14. The method according to any preceding claim, wherein the amplified product is detected within 15 minutes, less than 10 minutes, less than 9, 8, 7, 6, or even 5 minutes of the method commencing.

15. A binder DNA molecule for use in a method according to any preceding claim, the binder DNA molecule being capable of specifically binding an RNA template, the binder DNA molecule comprising:
a portion which is complementary to a target RNA molecule and which portion includes a DNA restriction endonuclease recognition site which may be nicked by a restriction endonuclease when the binder DNA is part of a DNA/RNA duplex; and
a further portion which is not complementary and does not bind to the target RNA molecule, when the portion which is complementary to a target RNA molecule is bound to the target RNA molecule.

16. The binder DNA according to claim 15, wherein the binder DNA is 20-50 nucleotides in length.

17. The binder DNA according to either of claim 15 or 16 wherein the region, which does not bind the target RNA sequence is 2-8 nucleotides in length and/or is at the 5′ end of the binder DNA.

18. The binder DNA according to any of claims 15-17 comprising one or more chemical modifications, optionally wherein said one or more chemical modifications is at the 3′ and/or 5′ end of the binder DNA.

19. The binder DNA molecule according to any of claims 15-18 wherein the binder DNA molecule consists essentially of, or consists of the sequence:
5′-AGG GTA AAC CAA ATA CCT GGT GTA TAC GTT-3′,
5′-AGG GTT AAA CCA CCG CCT GGA GAT CAA TTT-3′,
or
5′-AGG GTC CTT AAC TTG CCT GGT TGT GAT GGT-3′.

20. The binder DNA molecule according to claim 19, wherein the binder DNA consists essentially of, or consists of the sequence:
5′-AGG GTA AAC CAA ATA CCT GGT GTA TAC GTT-3′.

21. A kit for use in a method according to any of claims 1-14, the kit consisting essentially of, or consisting of:
a binder DNA molecule which is capable of specifically binding to the target RNA sequence at a first temperature, thereby forming an RNA/DNA duplex: the binder DNA molecule comprising a portion of sequence, which does not bind to the target RNA sequence at the first temperature and which comprises a sequence is complementary to a template DNA molecule; and a further portion of sequence, which is capable of specifically binding to the target RNA sequence at the first temperature and which includes the specific recognition sequence, or
a binder DNA molecule which is capable of specifically binding to the target RNA sequence at a first temperature, thereby forming an RNA/DNA duplex and capable of initiating chain elongation and generation of an extended RNA/DNA duplex in the presence of dNTPs and a reverse transcriptase, the extended RNA/DNA duplex comprising the specific recognition sequence, the kit optionally further comprising the reverse transcriptase; and
a restriction endonuclease which is capable of nicking the binder DNA sequence when bound to a target RNA molecule, at the recognition sequence of the restriction endonuclease.

22. The kit according to claim 21 wherein the binder DNA molecule is a binder DNA molecule according to any of claims 15-20.

23. The kit according to either of claim 21 or 22, wherein the restriction endonuclease is BstNI, AvrII or PhoI.
